(19) United States
(12) Patent Application Publication
(10) Pub. No.: US 2008/0058255 A1
(43) Pub. Date: Mar. 6, 2008

(54) Glycosylation-Disrupted Factor VII Variants

(75) Inventors: Gert Bolt, Vaerlose (DK); Thomas Dock Steenstrup, Gentofte (DK); Claus Kristensen, Bronshoj (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk HealthCare A/G, Zurich (CH)

(21) Appl. No.: 11/629,926

(22) PCT Filed: Jun. 17, 2005
(86) PCT No.: PCT/EP05/52834

§ 371 Date: Sep. 28, 2007

(30) Foreign Application Priority Data
Jun. 21, 2004 (DK) PA 2004 00967

(51) Int. Cl. A61K 38/00; A61P 7/00; C07K 14/745

(52) U.S. Cl. 514/12; 530/383

(57) ABSTRACT

The present invention relates to human coagulation Factor VII polypeptides, as well as polynucleotide constructs encoding such polypeptides, vectors and host cells comprising and expressing the polynucleotide, pharmaceutical compositions comprising Factor VII polypeptides, uses and methods of treatment; and any additional inventive features related thereto.

### FIELD OF THE INVENTION

The present invention relates to human coagulation Factor VII polypeptides, as well as polynucleotide constructs encoding such polypeptides, vectors and host cells comprising and expressing the polynucleotide, pharmaceutical compositions comprising Factor VII polypeptides, uses and methods of treatment; and any additional inventive features related thereto.

### BACKGROUND OF THE INVENTION

Blood coagulation is a process consisting of a complex interaction of various blood components (or factors) that eventually gives rise to a fibrin clot. Generally, the blood components, which participate in what has been referred to as the coagulation cascade, are enzymatically inactive proteins (proenzymes or zymogens) that are converted to proteolytic enzymes by the action of an activator (which itself is an activated clotting factor). Coagulation factors that have undergone such a conversion are generally referred to as “active factors”, and are designated by the addition of the letter “a” to the name of the coagulation factor (e.g. Factor VIIa).

Initiation of the haemostatic process is mediated by the formation of a complex between tissue factor, exposed as a result of injury to the vessel wall, and Factor VIIa. This complex then converts Factors IX and X to their active forms. Factor Xa converts limited amounts of prothrombin to thrombin on the tissue factor-bearing cell. Thrombin activates platelets and Factors V and VIII into Factors Va and VIIIa, both cofactors in the further process leading to the full thrombin burst. This process includes generation of Factor Xa by Factor IXa (in complex with factor VIIIa) and occurs on the surface of activated platelets. Thrombin finally converts fibrinogen to fibrin resulting in formation of a fibrin clot.

Factor VII is a trace plasma glycoprotein that circulates in blood as a single-chain zymogen. The zymogen is catalytically inactive. Single-chain Factor VII may be converted to two-chain Factor VIIa by Factor Xa, Factor XIIa, Factor IXa, Factor VIIa or thrombin in vitro. Factor Xa is believed to be the major physiological activator of Factor VII. The conversion of zymogen Factor VII into the activated two-chain molecule occurs by cleavage of an internal Arg₁₅₂-Ile₁₅₃ peptide bond.

It is often desirable to stimulate the coagulation cascade in a subject. Factor VIIa has been used to control bleeding disorders caused by, e.g., deficiency of a clotting factor (e.g. haemophilia A and B or deficiency of coagulation Factors XI or VII) or presence of clotting factor inhibitors. Factor VIIa has also been used to control excessive bleeding occurring in subjects with a normally functioning blood clotting cascade (no clotting factor deficiencies or inhibitors against any of the coagulation factors). Such bleeding may, for example, be caused by a defective platelet function, thrombocytopenia or von Willebrand's disease. Bleeding is also a major problem in connection with surgery and other forms of tissue damage or trauma.

There is a need in the art for Factor VII polypeptides having modified pharmacokinetic properties.

### SUMMARY OF THE INVENTION

The present invention provides variant Factor VII polypeptides in which at least one of the two N-linked glycosylation sites present in wild-type Factor VII has been disrupted.

In one aspect the present invention relates to a variant Factor VII polypeptide comprising at least one sequence alteration relative to the sequence of SEQ ID NO:1, wherein this alteration(s) is selected from the group consisting of:
(i) substitution of N145 with any other amino acid except A;
(ii) substitution of N322 with any other amino acid except A or D; and
(iii) substitution of N145 with any other amino acid except A and independent substitution of N322 for any other amino acid except A or D;
(iv) substitution of N145 with any other amino acid and independent substitution of N322 for any amino acid except for A or D; and
(v) substitution of N145 with any amino acid except A and independent substitution of N322 with any other amino acid.
(vi) substitution at any position relative to the sequence of SEQ ID NO:1, wherein said sequence alteration results in disruption of N-linked glycosylation at N145, N322, or both N145 and N322 and wherein said sequence alteration is not at positions 145 or 322.

In one series of embodiments, the variants comprise at least one sequence alteration relative to the sequence of SEQ ID NO:1 that involves: (i) substitution of N145 for any other amino acid except A; (ii) substitution of N322 for any other amino acid except A or D; or (iii) combinations of the foregoing. Non-limiting examples of such alterations include N145Q; N322Q; and N145Q/N322Q.

In another series of embodiments, the variants comprise at least one sequence alteration relative to the sequence of SEQ ID NO:1 that does not involve directly either position 145 or 322 and that, nonetheless, results in disruption of N-linked glycosylation at N145, N322, or both N145 and N322. Non-limiting examples of such alterations include (i) changing T147 to any other amino acid; changing S324 to any other amino acid; or independently changing both T147 and S324 to any other amino acid; (ii) changing A146, I323, or both to P; and (iii) changing K148, E325, or both to P; (or any other amino acid that will result in disruption of glycosylation at the cognate site); and (iv) insertion or deletion of one or more amino acids between N145-T147 and/or between N322-S324, when such insertion or deletion results in disruption of glycosylation at the cognate site.

In one aspect, the invention provides pharmaceutical formulations comprising glycosylation-disrupted Factor VIIa variant polypeptides and a pharmaceutically acceptable carrier or excipient.

In another aspect, the invention provides methods for treating a Factor VIIa-responsive syndrome, which are carried out by administering to a patient in need of such treatment a therapeutically effective amount of a glycosylation-disrupted Factor VIIa variant polypeptide.

In another aspect, the invention provides kits that may be used for treating Factor VIIa-responsive syndromes that comprise therapeutically effective amounts of a glycosylation-disrupted Factor VIIa variant polypeptide.

### DETAILED DESCRIPTION OF THE INVENTION

The present invention relates to glycosylation-disrupted Factor VII polypeptides, that is, Factor VII polypeptides lacking one or both of the N-linked oligosaccharides that are present in wild-type Factor VII. The present inventors have surprisingly found that Factor VIIa polypeptides lacking either or both of the normal N-linked oligosaccharide moieties exhibit enhanced Factor VIIa biological activity. The Factor VII polypeptides of the present invention provide an alternative to wild-type Factor VIIa for procoagulant therapy and other uses.

Also the faster clearance of these glycosylation-disrupted Factor VII polypeptides could be an advantage in some therapeutic applications.

Furthermore, they offer advantages over wild-type glycosylated Factor VIIa, such as, e.g., by allowing the use of an expanded range of expression systems in which they can be produced.

Wild-type Factor VII refers to a polypeptide having the amino acid sequence disclosed in U.S. Pat. No. 4,784,950 (SEQ ID NO:1). The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa.

Factor VII variants are polypeptides having a sequence derived from SEQ ID NO:1 by substitution, deletion, and/or insertion of one more amino acids. Insertion may take place either at the N-terminal end, C-terminal, and/or internally. In designating amino acid substitutions, the first letter represents the amino acid naturally present at a position of human wild type FVII. The following number represents the position in human wild type FVII. The second letter represent the amino acid replacing the natural amino acid.

The present invention provides Factor VII variants having a glycosylation-disrupting substitution at either N145 or N322, or at both N145 and N322. In one series of embodiments, N145 is substituted by any amino acid (naturally occurring or non-naturally occurring) except for alanine (A). In another series of embodiments, N322 is substituted by any amino acid (naturally occurring or non-naturally occurring) except for A or aspartic acid (D). In another series of embodiments, N145 is substituted by any amino acid except for A and N322 is substituted by any amino acid except for A or D. In another series of embodiments, N145 is substituted with any amino acid (naturally occurring or non-naturally occurring) and N322 is substituted by any amino acid except for A or D. In another series of embodiments, N145 is substituted with any amino acid except Ala and N322 is substituted with any amino acid. In another series of embodiments, N145 and N322 are each independently substituted with any amino acid.

In one series of embodiments, the invention encompasses Factor VII variants comprising N145Q or N322Q or the combination N145Q/N322Q. The invention also encompasses Factor VII variants in which any of residues 145-147 and/or residues 322-324 have been eliminated (i.e., deleted and not substituted with any another amino acid).

In one series of embodiments, the invention encompasses Factor VII variants comprising at least one sequence alteration, wherein the alteration which results in disruption of N-linked glycosylation is at position 146, 323, or both 146 and 323 relative to the sequence of SEQ ID NO:1.

In one series of embodiments, the invention encompasses Factor VII variants comprising at least one sequence alteration, wherein the alteration which results in disruption of N-linked glycosylation is at position 147, 324, or both 327 and 324 relative to the sequence of SEQ ID NO:1.

In one series of embodiments, the invention encompasses Factor VII variants comprising at least one sequence alteration, wherein the alteration which results in disruption of N-linked glycosylation is at position 148, 325, or both 148 and 325 relative to the sequence of SEQ ID NO:1.

In one series of embodiments, the invention encompasses variant Factor VII polypeptides comprising at least one sequence alteration, wherein the alterations which results in disruption of N-linked glycosylation are selected from K143N/N145T and K143N/N145T/R315N/V317T.

In one series of embodiments, the invention encompasses Factor VII variants comprising at least one sequence alteration which results in disruption of N-linked glycosylation further comprising a sequence alteration selected from the group consisting of: R152E; S344A; L305V; L305V/M306D/D309S; L305I, L305T, F374P, V158T/M298Q, V158D/E296V/M298Q, K337A, M298Q, V158D/M298Q, L305V/K337A, V158D/E296V/M298Q/L305V, V158D/E296V/M298Q/K337A, V158D/E296V/M298Q/L305V/K337A, K157A, E296V, E296V/M298Q, V158D/E296V, V158D/M298K, and S336G, L305V/K337A, L305V/V158D, L305V/E296V, L305V/M298Q, L305V/V158T, L305V/K337A/V158T, L305V/K337A/M298Q, L305V/K337A/E296V, L305V/K337A/V158D, L305V/V158D/M298Q, L305V/V158D/E296V, L305V/V158T/M298Q, L305V/V158T/E296V, L305V/E296V/M298Q, L305V/V158D/E296V/M298Q, L305V/V158T/E296V/M298Q, L305V/V158T/K337A/M298Q, L305V/V158T/E296V/K337A, L305V/V158D/K337A/M298Q, L305V/V158D/E296V/K337A-FVII, L305V/V158D/E296V/M298Q/K337A, L305V/V158T/E296V/M298Q/K337A, S314E/K316H, S314E/K316Q, S314E/L305V, S314E/K337A, S314E/V158D, S314E/E296V, S314E/M298Q, S314E/V158T, K316H/L305V, K316H/K337A, K316H/V158D, K316H/E296V, K316H/M298Q, K316H/V158T, K316Q/L305V, K316Q/K337A, K316Q/V158D, K316Q/E296V, K316Q/M298Q, K316Q/V158T, S314E/L305V/K337A, S314E/L305V/V158D, S314E/L305V/E296V, S314E/L305V/M298Q, S314E/L305V/V158T, S314E/L305V/K337A/V158T, S314E/L305V/K337A/M298Q, S314E/L305V/K337A/E296V, S314E/L305V/K337A/V158D, S314E/L305V/V158D/M298Q, S314E/L305V/V158D/E296V, S314E/L305V/V158T/M298Q, S314E/L305V/V158T/E296V, S314E/L305V/E296V/M298Q, S314E/L305V/V158D/E296V/M298Q, S314E/L305V/V158T/E296V/M298Q, S314E/L305V/V158T/K337A/M298Q, S314E/L305V/V158T/E296V/K337A, S314E/L305V/V158D/K337A/M298Q, S314E/L305V/V158D/E296V/K337A, S314E/L305V/V158D/E296V/M298Q/K337A, S314E/L305V/V158T/E296V/M298Q/K337A, K316H/L305V/K337A, K316H/L305V/V158D, K316H/L305V/E296V, K316H/L305V/M298Q, K316H/L305V/V158T, K316H/L305V/K337A/V158T, K316H/L305V/K337A/M298Q, K316H/L305V/K337A/E296V, K316H/L305V/K337A/V158D, K316H/L305V/V158D/M298Q, K316H/L305V/V158D/E296V, K316H/L305V/V158T/M298Q, K316H/L305V/V158T/E296V, K316H/L305V/E296V/M298Q, K316H/L305V/V158D/E296V/M298Q, K316H/L305V/V158T/E296V/M298Q, K316H/L305V/V158T/K337A/M298Q, K316H/L305V/V158T/E296V/K337A, K316H/L305V/V158D/K337A/M298Q, K316H/L305V/V158D/E296V/K337A, K316H/L305V/V158D/E296V/M298Q/K337A, K316H/L305V/V158T/E296V/M298Q/K337A, K316Q/L305V/K337A, K316Q/L305V/V158D, K316Q/L305V/E296V, K316Q/L305V/M298Q, K316Q/L305V/V158T, K316Q/L305V/K337A/V158T, K316Q/L305V/K337A/M298Q, K316Q/L305V/K337A/E296V, K316Q/L305V/K337A/V158D, K316Q/L305V/V158D/M298Q, K316Q/L305V/V158D/E296V, K316Q/L305V/V158T/M298Q, K316Q/L305V/V158T/E296V, K316Q/L305V/E296V/M298Q, K316Q/L305V/V158D/E296V/M298Q, K316Q/L305V/V158T/E296V/M298Q, K316Q/L305V/V158T/K337A/M298Q, K316Q/L305V/V158T/E296V/K337A, K316Q/L305V/V158D/K337A/M298Q, K316Q/L305V/V158D/E296V/K337A, K316Q/L305V/V158D/E296V/M298Q/K337A, K316Q/L305V/V158T/E296V/M298Q/K337A, F374Y/K337A, F374Y/V158D, F374Y/E296V, F374Y/M298Q, F374Y/V158T, F374Y/S314E, F374Y/L305V, F374Y/L305V/K337A, F374Y/L305V/V158D, F374Y/L305V/E296V, F374Y/L305V/M298Q, F374Y/L305V/V158T, F374Y/L305V/S314E, F374Y/K337A/S314E, F374Y/K337A/V158T, F374Y/K337A/M298Q, F374Y/K337A/E296V, F374Y/K337A/V158D, F374Y/V158D/S314E, F374Y/V158D/M298Q, F374Y/V158D/E296V, F374Y/V158T/S314E, F374Y/V158T/M298Q, F374Y/V158T/E296V, F374Y/E296V/S314E, F374Y/S314E/M298Q, F374Y/E296V/M298Q, F374Y/L305V/K337A/V158D, F374Y/L305V/K337A/E296V, F374Y/L305V/K337A/M298Q, F374Y/L305V/K337A/V158T, F374Y/L305V/K337A/S314E, F374Y/L305V/V158D/E296V, F374Y/L305V/V158D/M298Q, F374Y/L305V/V158D/S314E, F374Y/L305V/E296V/M298Q, F374Y/L305V/E296V/V158T, F374Y/L305V/E296V/S314E, F374Y/L305V/M298Q/V158T, F374Y/L305V/M298Q/S314E, F374Y/L305V/V158T/S314E, F374Y/K337A/S314E/V158T, F374Y/K337A/S314E/M298Q, F374Y/K337A/S314E/E296V, F374Y/K337A/S314E/V158D, F374Y/K337A/V158T/M298Q, F374Y/K337A/V158T/E296V, F374Y/K337A/M298Q/E296V, F374Y/K337A/M298Q/V158D, F374Y/K337A/E296V/V158D, F374Y/V158D/S314E/M298Q, F374Y/V158D/S314E/E296V, F374Y/V158D/M298Q/E296V, F374Y/V158T/S314E/E296V, F374Y/V158T/S314E/M298Q, F374Y/V158T/M298Q/E296V, F374Y/E296V/S314E/M298Q, F374Y/L305V/M298Q/K337A/S314E, F374Y/L305V/E296V/K337A/S314E, F374Y/E296V/M298Q/K337A/S314E, F374Y/L305V/E296V/M298Q/K337A, F374Y/L305V/E296V/M298Q/S314E, F374Y/V158D/E296V/M298Q/K337A, F374Y/V158D/E296V/M298Q/S314E, F374Y/L305V/V158D/K337A/S314E, F374Y/V158D/M298Q/K337A/S314E, F374Y/V158D/E296V/K337A/S314E, F374Y/L305V/V158D/E296V/M298Q, F374Y/L305V/V158D/M298Q/K337A, F374Y/L305V/V158D/E296V/K337A, F374Y/L305V/V158D/M298Q/S314E, F374Y/L305V/V158D/E296V/S314E, F374Y/V158T/E296V/M298Q/K337A, F374Y/V158T/E296V/M298Q/S314E, F374Y/L305V/V158T/K337A/S314E, F374Y/V158T/M298Q/K337A/S314E, F374Y/V158T/E296V/K337A/S314E, F374Y/L305V/V158T/E296V/M298Q, F374Y/L305V/V158T/M298Q/K337A, F374Y/L305V/V158T/E296V/K337A, F374Y/L305V/V158T/M298Q/S314E, F374Y/L305V/V158T/E296V/S314E, F374Y/E296V/M298Q/K337A/V158T/S314E, F374Y/V158D/E296V/M298Q/K337A/S314E, F374Y/L305V/V158D/E296V/M298Q/S314E, F374Y/L305V/E296V/M298Q/V158T/S314E, F374Y/L305V/E296V/M298Q/K337A/V158T, F374Y/L305V/E296V/K337A/V158T/S314E, F374Y/L305V/M298Q/K337A/V158T/S314E, F374Y/L305V/V158D/E296V/M298Q/K337A, F374Y/L305V/V158D/E296V/K337A/S314E, F374Y/L305V/V158D/M298Q/K337A/S314E, F374Y/L305V/E296V/M298Q/K337A/V158T/S314E, F374Y/L305V/V158D/E296V/M298Q/K337A/S314E; R152E, S344A; P11Q/K33E, T106N, V253N, R290N/A292T, G291N, R315N/V317T, and K143N/R315N/V317T.

In one series of embodiments, the invention encompasses variant Factor VII polypeptides, wherein said polypeptide has a decreased half-life as compared to wild type human FVIIa.

In some embodiments, the Factor VII variants of the present invention exhibit a decrease in half-life of at least about 0.25 h, such as at least about 0.5 h, more such as at least about 1 h, such as at least about 2 h, as measured in human plasma relative to the half-life of wild-type Factor VII.

In some embodiment the variant Factor VII polypeptide exhibit a half-life as measured in human plasma lower than about 2 h, such as lower than about 1.5 h, such as lower than about 1 h, such as lower than 30 min.

The following table further illustrates different non-limiting embodiments of the present invention:

| Sequence at 145 | Sequence at 322 |
|---|---|
| R | N |
| D | N |
| C | N |
| E | N |
| Q | N |
| G | N |
| H | N |
| I | N |
| L | N |
| K | N |
| M | N |
| F | N |
| P | N |
| S | N |
| T | N |
| W | N |
| Y | N |
| V | N |
| N | A |
| N | R |
| N | C |
| N | E |
| N | Q |
| N | G |
| N | H |
| N | I |
| N | L |
| N | K |
| N | M |
| N | F |
| N | P |
| N | S |
| N | T |
| N | W |
| N | Y |
| N | V |

It will be understood that any of the substitutions listed above for N145 may be combined with any of the substitutions listed above for N322.

The invention also encompasses disruption of glycosylation at N145 and/or N322 by substitution of the N+1, N+2, or N+3 residue. That is, polypeptides having a substitution of S147 to any residue other than T and/or substitution of T324 to any residue other than S, as well as polypeptides in which one or more of A146, K148, I323, and E325 have been substituted by a glycosylation-disrupting amino acid (exemplified by, but not limited to, proline (P)) are also encompassed by the present invention.

The invention also encompasses insertion of one or more amino acids between N145 and A146; between A146 and S147; between N322 and I323; or between I323 and T324 of SEQ ID NO1, when such insertion results in a disruption of glycosylation at N145 or N322, as applicable.

It will be understood that, in Factor VII variants in which glycosylation is disrupted at both N145 and N322, any combination of the above-disclosed means may be used (such as, e.g., substitution of N145 combined with substitution at T324, or any other useful combination.)

In one series of embodiments, modifications of the amino acid sequence of Factor VIIa that disrupt glycosylation at N145, N322, or both, are accompanied by one or more additional sequence alterations within the Factor VII sequence.

Non-limiting examples of additional modifications include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII variants as disclosed in PCT/DK02/00189 (corresponding to WO 02/077218); and FVII variants exhibiting increased proteolytic stability as disclosed in WO 02/38162 (Scripps Research Institute); FVII variants having a modified Gla-domain and exhibiting an enhanced membrane binding as disclosed in WO 99/20767, US patents U.S. Pat. No. 6,017,882 and U.S. Pat. No. 6,747,003, US patent application 20030100506 (University of Minnesota) and WO 00/66753, US patent applications US 20010018414, US 2004220106, and US 200131005, US patents U.S. Pat. No. 6,762,286 and U.S. Pat. No. 6,693,075 (University of Minnesota); and FVII variants as disclosed in WO 01/58935, U.S. patent U.S. Pat. No. 6,806,063, US patent application 20030096338 (Maxygen ApS), WO 03/93465 (Maxygen ApS), WO 04/029091 (Maxygen ApS), WO 04/083361 (Maxygen ApS), and WO 04/111242 (Maxygen ApS), as well as in WO 04/108763 (Canadian Blood Services).

Non-limiting examples of FVII variants having increased biological activity compared to wild-type FVIIa include FVII variants as disclosed in WO 01/83725, WO 02/22776, WO 02/077218, PCT/DK02/00635 (corresponding to WO 03/027147), Danish patent application PA 2002 01423 (corresponding to WO 04/029090), Danish patent application PA 2001 01627 (corresponding to WO 03/027147); WO 02/38162 (Scripps Research Institute); and FVIIa variants with enhanced activity as disclosed in JP 2001061479 (Chemo-Sero-Therapeutic Res Inst.).

Further non-limiting examples of additional modifications include: R152E; S344A; L305V; L305V/M306D/D3095; L305I, L305T, F374P, V158T/M298Q, V158D/E296V/M298Q, K337A, M298Q, V158D/M298Q, L305V/K337A, V158D/E296V/M298Q/L305V, V158D/E296V/M298Q/K337A, V158D/E296V/M298Q/L305V/K337A, K157A, E296V, E296V/M298Q, V158D/E296V, V158D/M298K, and S336G, L305V/K337A, L305V/V158D, L305V/E296V, L305V/M298Q, L305V/V158T, L305V/K337A/V158T, L305V/K337A/M298Q, L305V/K337A/E296V, L305V/K337A/V158D, L305V/V158D/M298Q, L305V/V158D/E296V, L305V/V158T/M298Q, L305V/V158T/E296V, L305V/E296V/M298Q, L305V/V158D/E296V/M298Q, L305V/V158T/E296V/M298Q, L305V/V158T/K337A/M298Q, L305V/V158T/E296V/K337A, L305V/V158D/K337A/M298Q, L305V/V158D/E296V/K337A-FVII, L305V/V158D/E296V/M298Q/K337A, L305V/V158T/E296V/M298Q/K337A, S314E/K316H, S314E/K316Q, S314E/L305V, S314E/K337A, S314E/V158D, S314E/E296V, S314E/M298Q, S314E/V158T, K316H/L305V, K316H/K337A, K316H/V158D, K316H/E296V, K316H/M298Q, K316H/V158T, K316Q/L305V, K316Q/K337A, K316Q/V158D, K316Q/E296V, K316Q/M298Q, K316Q/V158T, S314E/L305V/K337A, S314E/L305V/V158D, S314E/L305V/E296V, S314E/L305V/M298Q, S314E/L305V/V158T, S314E/L305V/K337A/V158T, S314E/L305V/K337A/M298Q, S314E/L305V/K337A/E296V, S314E/L305V/K337A/V158D, S314E/L305V/V158D/M298Q, S314E/L305V/V158D/E296V, S314E/L305V/V158T/M298Q, S314E/L305V/V158T/E296V, S314E/L305V/E296V/M298Q, S314E/L305V/V158D/E296V/M298Q, S314E/L305V/V158T/E296V/M298Q, S314E/L305V/V158T/K337A/M298Q, S314E/L305V/V158T/E296V/K337A, S314E/L305V/V158D/K337A/M298Q, S314E/L305V/V158D/E296V/K337A, S314E/L305V/V158D/E296V/M298Q/K337A, S314E/L305V/V158T/E296V/M298Q/K337A, K316H/L305V/K337A, K316H/L305V/V158D, K316H/L305V/E296V, K316H/L305V/M298Q, K316H/L305V/V158T, K316H/L305V/K337A/V158T, K316H/L305V/K337A/M298Q, K316H/L305V/K337A/E296V, K316H/L305V/K337A/V158D, K316H/L305V/V158D/M298Q, K316H/L305V/V158D/E296V, K316H/L305V/V158T/M298Q, K316H/L305V/V158T/E296V, K316H/L305V/E296V/M298Q, K316H/L305V/V158D/E296V/M298Q, K316H/L305V/V158T/E296V/M298Q, K316H/L305V/V158T/K337A/M298Q, K316H/L305V/V158T/E296V/K337A, K316H/L305V/V158D/K337A/M298Q, K316H/L305V/V158D/E296V/K337A, K316H/L305V/V158D/E296V/M298Q/K337A, K316H/L305V/V158T/E296V/M298Q/K337A, K316Q/L305V/K337A, K316Q/L305V/V158D, K316Q/L305V/E296V, K316Q/L305V/M298Q, K316Q/L305V/V158T, K316Q/L305V/K337A/V158T, K316Q/L305V/K337A/M298Q, K316Q/L305V/K337A/E296V, K316Q/L305V/K337A/V158D, K316Q/L305V/V158D/M298Q, K316Q/L305V/V158D/E296V, K316Q/L305V/V158T/M298Q, K316Q/L305V/V158T/E296V, K316Q/L305V/E296V/M298Q, K316Q/L305V/V158D/E296V/M298Q, K316Q/L305V/V158T/E296V/M298Q, K316Q/L305V/V158T/K337A/M298Q, K316Q/L305V/V158T/E296V/K337A, K316Q/L305V/V158D/K337A/M298Q, K316Q/L305V/V158D/E296V/K337A, K316Q/L305V/V158D/E296V/M298Q/K337A, K316Q/L305V/V158T/E296V/M298Q/K337A, F374Y/K337A, F374Y/V158D, F374Y/E296V, F374Y/M298Q, F374Y/V158T, F374Y/S314E, F374Y/L305V, F374Y/L305V/K337A, F374Y/L305V/V158D, F374Y/L305V/E296V, F374Y/L305V/M298Q, F374Y/L305V/V158T, F374Y/L305V/S314E, F374Y/K337A/S314E, F374Y/K337A/V158T, F374Y/K337A/M298Q, F374Y/K337A/E296V, F374Y/K337A/V158D, F374Y/V158D/S314E, F374Y/V158D/M298Q, F374Y/V158D/E296V, F374Y/V158T/S314E, F374Y/V158T/M298Q, F374Y/V158T/E296V, F374Y/E296V/S314E, F374Y/S314E/M298Q, F374Y/E296V/M298Q, F374Y/L305V/K337A/V158D, F374Y/L305V/K337A/E296V, F374Y/L305V/K337A/M298Q, F374Y/L305V/K337A/V158T, F374Y/L305V/K337A/S314E, F374Y/L305V/V158D/E296V, F374Y/L305V/V158D/M298Q, F374Y/L305V/V158D/S314E, F374Y/L305V/E296V/M298Q, F374Y/L305V/E296V/V158T, F374Y/L305V/E296V/S314E, F374Y/L305V/M298Q/V158T, F374Y/L305V/M298Q/S314E, F374Y/L305V/V158T/S314E, F374Y/K337A/S314E/V158T, F374Y/K337A/S314E/M298Q, F374Y/K337A/S314E/E296V, F374Y/K337A/S314E/V158D, F374Y/K337A/V158T/M298Q, F374Y/K337A/V158T/E296V, F374Y/K337A/M298Q/E296V, F374Y/K337A/M298Q/V158D, F374Y/K337A/E296V/V158D, F374Y/V158D/S314E/M298Q, F374Y/V158D/S314E/E296V, F374Y/V158D/M298Q/E296V, F374Y/V158T/S314E/E296V, F374Y/V158T/S314E/M298Q, F374Y/V158T/M298Q/E296V, F374Y/E296V/S314E/M298Q, F374Y/L305V/M298Q/K337A/S314E, F374Y/L305V/E296V/K337A/S314E, F374Y/E296V/M298Q/K337A/S314E, F374Y/L305V/E296V/M298Q/K337A, F374Y/L305V/E296V/M298Q/S314E, F374Y/V158D/E296V/M298Q/K337A, F374Y/V158D/E296V/M298Q/S314E, F374Y/L305V/V158D/K337A/S314E, F374Y/V158D/M298Q/K337A/S314E, F374Y/V158D/E296V/K337A/S314E, F374Y/L305V/V158D/E296V/M298Q, F374Y/L305V/V158D/M298Q/K337A, F374Y/L305V/V158D/E296V/K337A, F374Y/L305V/V158D/M298Q/S314E, F374Y/L305V/V158D/E296V/S314E, F374Y/V158T/E296V/M298Q/K337A, F374Y/V158T/E296V/M298Q/S314E, F374Y/L305V/V158T/K337A/S314E, F374Y/V158T/M298Q/K337A/S314E, F374Y/V158T/E296V/K337A/S314E, F374Y/L305V/V158T/E296V/M298Q, F374Y/L305V/V158T/M298Q/K337A, F374Y/L305V/V158T/E296V/K337A, F374Y/L305V/V158T/M298Q/S314E, F374Y/L305V/V158T/E296V/S314E, F374Y/E296V/M298Q/K337A/V158T/S314E, F374Y/V158D/E296V/M298Q/K337A/S314E, F374Y/L305V/V158D/E296V/M298Q/S314E, F374Y/L305V/E296V/M298Q/V158T/S314E, F374Y/L305V/E296V/M298Q/K337A/V158T, F374Y/L305V/E296V/K337A/V158T/S314E, F374Y/L305V/M298Q/K337A/V158T/S314E, F374Y/L305V/V158D/E296V/M298Q/K337A, F374Y/L305V/V158D/E296V/K337A/S314E, F374Y/L305V/V158D/M298Q/K337A/S314E, F374Y/L305V/E296V/M298Q/K337A/V158T/S314E, F374Y/L305V/V158D/E296V/M298Q/K337A/S314E, R152E, S344A; P11Q/K33E, T106N, V253N, R290N/A292T, G291N, R315N/V317T, K143N/R315N/V317T; and FVII having substitutions, additions or deletions in the amino acid sequence from T233 to N240, and FVII having substitutions, additions or deletions in the amino acid sequence from R304 to C329 (other than 322-324, which are specified above). In one series of embodiments, the variants of the invention comprise K143N/N145T; in some embodiments, they comprise K143N/N145T/R315N/V317T.

The present invention also encompasses fragments of Factor VII that comprise residues 145, 322, or both, and in which glycosylation-disrupting alterations have been introduced; such as, for example, peptides that might be part of a fusion protein (i.e., in combination with other non-Factor VII sequences.) In some embodiments, the Factor VII-derived fragments retain Factor VII biological activity. In some embodiments, the Factor VII-derived fragments retain Factor VII host-dependent immunogenicity/antigenicity, or lack thereof.

The present invention also encompasses Factor VII polypeptides in which modifications of the amino acid sequence of Factor VIIa that disrupt glycosylation at N145, N322, or both, are accompanied by chemical modification of one or more of the amino acids of the polypeptide, such as, e.g. by alkylation, PEGylation, acylation, ester formation, amide formation, or the like. Non-limiting examples of such Factor VII derivatives include PEGylated Factor VIIa, cysteine-PEGylated Factor VIIa, and variants thereof.

This includes but is not limited to PEGylated human Factor VIIa, cysteine-PEGylated human Factor VIIa and variants thereof. Non-limiting examples of Factor VII derivatives includes GlycoPegylated FVII derivatives as disclosed in WO 03/31464 and US Patent applications US 20040043446, US 20040063911, US 20040142856, US 20040137557, and US 20040132640 (Neose Technologies, Inc.); FVII conjugates as disclosed in WO 01/04287, US patent application 20030165996, WO 01/58935, WO 03/93465 (Maxygen ApS) and WO 02/02764, US patent application 20030211094 (University of Minnesota).

PEGylation refers to conjugation of a PEG molecule to any part of the Factor VIIa polypeptide, including, but not limited to, any amino acid residue or carbohydrate moiety. The term “cysteine-PEGylated human Factor VIIa” means Factor VIIa having a PEG molecule conjugated to a sulfhydryl group of a cysteine introduced in Factor VIIa. Other non-limiting examples of modified Factor VIIa include Factor VIIa that has been proteolytically cleaved between residues 290 and 291 or between residues 315 and 316 (Mollerup et al., Biotechnol. Bioeng. 48:501-505, 1995); and oxidized forms of Factor VIIa (Kornfelt et al., Arch. Biochem. Biophys. 363:43-54, 1999).

The present invention also encompasses methods for identifying useful glycosylation-disrupted variants of Factor VII. These methods are carried out by the steps of:

(a) obtaining the expression products of a plurality of DNA species encoding different glycosylation-disrupted variants of Factor VII (such as, e.g., variants in which glycosylation at N145, N322, or both is disrupted), under conditions that produce activated forms of the expression products; and

(b) testing the expression products for one or more of: Factor VIIa biological activity; bioavailability; storage stability; and immunogenicity (see below).

In some embodiments, prior to step (b), the expression products may be subjected to chemical modifications according to the invention (such as, e.g., further proteolytic cleavage, PEGylation, and the like.)

In some embodiments, particular sequence modifications that disrupt glycosylation at N145 may be combined with other sequence modifications that disrupt glycosylation at N322, and the method may further comprise repeating steps (a) and (b) until a desired profile of bioactivity, bioavailability, and/or storage stability is achieved.

Factor VIIa Properties

In some embodiments, the Factor VII/VIIa glycosylation-disrupted variants of the present invention exhibit improved biological activity, pharmacokinetic properties, and/or storage stability compared with unmodified (wild-type) Factor VII/Factor VIIa.

For purposes of the invention, Factor VIIa biological activity may be quantified by measuring the ability of a preparation to promote blood clotting using Factor VII-deficient plasma and thromboplastin, as described, e.g., in U.S. Pat. No. 5,997,864. In this assay, biological activity is expressed as the reduction in clotting time relative to a control sample and is converted to “Factor VII units” by comparison with a pooled human serum standard containing 1 unit/ml Factor VII activity. Alternatively, clot lysis time and clot strength may be measured by thromboelastograpy as described by, e.g., Vig et al. (2001) Blood coagulation & fibrinolysis, Vol. 12 (7) pp. 555-561. and Sorensen (2003) Throm Haemost 1:551-558. Alternatively, clot strength may be assayed as described by Carr et al, (1991), Am. J. Med. Sci. 302: 13-8. One parameter that reflects the clotting activity of Factor VIIa as measured by thromboelastography is the “overall clot quality” (OCQ). Once clot formation has been initiated (t=0), measurement of the clot strength as a function of time reveals a maximum velocity (max vel) of clot formation as well as the time required to reach the maximum velocity (t_{max vel}). Subsequently, addition of tissue plasminogen activator (TPA) allows measurement of fibrinolysis and derivation of the time required to reach the maximum velocity of fibrinolysis (t_{min vel}). OCQ is calculated as:
(Max vel/t_{max vel})×(t_{min vel}−t_{max vel}).

Factor VIIa biological activity may also be quantified by (i) measuring the ability of Factor VIIa to produce Factor Xa in a system comprising TF embedded in a lipid membrane and Factor X. (Persson et al., J. Biol. Chem. 272:19919-19924, 1997); (ii) measuring Factor X hydrolysis in an aqueous system; (iii) measuring its physical binding to TF using an instrument based on surface plasmon resonance (Persson, FEBS Letts. 413:359-363, 1997) (iv) measuring hydrolysis of a synthetic substrate; and/or (v) measuring generation of thrombin in a TF-independent in vitro system.

Factor VII variants having improved or enhanced biological activity relative to wild-type Factor VIIa encompass those that exhibit at least about 125%, such as at least about 150%, such as at least about 175%, such as at least about 200% of the specific activity of wild-type Factor VIIa that has been produced in the same cell type, when tested in one or more of a clotting assay, proteolysis assay, or TF binding assay as described above.

Factor VIIa variants of particular interest are variants in which the ratio between the activity of the variant and the activity of wild type Factor VII is above 1.0, e.g. at least about 1.25, such as, at least about 1.5, 1.75, 2.0, 2.5, or 3.0.

Bioavailability refers to the proportion of an administered dose of a Factor VII variant polypeptide that can be detected in plasma at predetermined times after administration. Typically, bioavailability is measured in test animals by administering a dose of between about 25-250 μg/kg of the preparation; obtaining plasma samples at predetermined times after administration; and determining the content of the Factor VII variant in the samples using one or more of a clotting assay (or any bioassay), an immunoassay, or an equivalent. The data are typically displayed graphically as [Factor VII] v. time and the bioavailability is expressed as the area under the curve (AUC). Relative bioavailability of a Factor VII variant refers to the ratio between the AUC of the variant and that of wild-type Factor VII.

In some embodiments, the Factor VII variants of the present invention exhibit a relative bioavailability of at least about 110%, such as at least about 120%, such as at least about 130%, such as at least about 140% of the bioavailability of wild-type Factor VII. The bioavailability may be measured in any mammalian species, such as dogs, and the predetermined times used for calculating AUC may encompass different increments from 10 min-8 h.

“Half-life” refers to the time required for the plasma concentration of Factor VII variant polypeptides to decrease from a particular value to half of that value. Half-life may be determined using the same procedure as for bioavailability.

In one series of embodiments, the invention encompasses variant Factor VII polypeptides, wherein said polypeptide has a decreased half-life as compared to wild type human FVIIa.

In some embodiments, the Factor VII variants of the present invention exhibit an increase in half-life of at least about 0.25 h, such as at least about 0.5 h, such as at least about 1 h, such as at least about 2 h, as measured in human plasma relative to the half-life of wild-type human Factor VII.

Immunogenicity of a preparation refers to the ability of the preparation, when administered to a human, to elicit a deleterious immune response, whether humoral, cellular, or both. Immunogenicity may be measured by quantifying the presence of anti-Factor VII antibodies and/or Factor VII-responsive T-cells in a sensitive individual, using conventional methods known in the art. In some embodiments, the Factor VII variants of the present invention exhibit a decrease in immunogenicity in a sensitive individual of at least about 10%, such as at least about 25%, such as at least about 40%, such as at least about 50%, relative to the immunogenicity for that individual of exogenously administered wild-type Factor VII.

Storage stability of a Factor VII preparation may be assessed by measuring (a) the time required for 20% of the bioactivity of a preparation to decay when stored as a dry powder at 25° C. and/or (b) the time required for a doubling in the proportion of Factor VIIa aggregates in the preparation.

In some embodiments, the Factor VII variants of the invention exhibit an increase of at least about 30%, such as at least about 60%, such as at least about 100%, in the time required for 20% of the bioactivity to decay relative to the time required for the same phenomenon with wild-type Factor VII, when both preparations are stored as dry powders at 25° C.

In some embodiments, the Factor VII variants of the invention exhibit an increase of at least about 30%, such as at least about 60%, such as at least about 100%, in the time required for doubling of aggregates relative to wild-type Factor VII, when both preparations are stored as dry powders at 25° C. The content of aggregates is determined by gel permeation HPLC on a Protein Pak 300 SW column (7.5×300 mm) (Waters, 80013) as follows. The column is equilibrated with Eluent A (0.2 M ammonium sulfate, 5% isopropanol, pH adjusted to 2.5 with phosphoric acid, and thereafter pH is adjusted to 7.0 with triethylamine), after which 25 μg of sample is applied to the column. Elution is with Eluent A at a flow rate of 0.5 ml/min for 30 min, and detection is achieved by measuring absorbance at 215 nm. The content of aggregates is calculated as the peak area of the Factor VII aggregates/total area of Factor VII peaks (monomer and aggregates).

DNA Constructs, Vectors, and Expression of Glycosylation-Disrupted Factor VII Variants in Recombinant Host Cells

The present invention provides polynucleotide constructs encoding the glycosylation-disrupted Factor VII variants disclosed herein; vectors comprising the constructs, and host cells comprising the vectors in which the variants may be expressed. The variants may be constructed from wild-type Factor VII-encoding DNA using any conventional method. Expression vectors for use in expressing Factor VIIa polypeptide variants will comprise a promoter capable of directing the transcription of a cloned gene or cDNA. The promoter may be any DNA sequence that exhibits transcriptional activity in the host cell of choice and may be derived from genes encoding proteins either homologous or heterologous to the host cell

To direct the Factor VII variants of the present invention into the secretory pathway of the host cells, a secretory signal sequence (also known as a leader sequence or pre sequence) may be provided in the recombinant vector. The secretory signal sequence is joined to the DNA sequences encoding the human Factor VII polypeptide variants in the correct reading frame. Secretory signal sequences are commonly positioned 5′ to the DNA sequence encoding the peptide. The secretory signal sequence may be that normally associated with the protein, may be from a gene encoding another secreted protein, or may be a synthetic peptide.

For secretion from yeast cells, suitable signal peptides include, without limitation, the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137). Furthermore, a sequence encoding a leader peptide may also be inserted downstream of the signal sequence and upstream of the DNA sequence encoding the human Factor VII variants. The function of the leader peptide is to allow the expressed peptide to be directed from the endoplasmic reticulum to the Golgi apparatus and further to a secretory vesicle for secretion into the culture medium (i.e. exportation of the human Factor VII polypeptide variants across the cell wall or at least through the cellular membrane into the periplasmic space of the yeast cell). In one embodiment, the leader peptide is the yeast alpha-factor leader (the use of which is described in e.g. U.S. Pat. No. 4,546,082, U.S. Pat. No. 4,870,008, EP 16 201, EP 123 294, EP 123 544 and EP 163 529). Alternatively, the leader peptide may be a synthetic leader peptide, i.e., a leader peptide not found in nature. Synthetic leader peptides may, for instance, be constructed as described in WO 89/02463 or WO 92/11378.

The procedures used to ligate the DNA sequences coding for Factor VII variants, a promoter and optionally a secretory signal sequence and/or a terminator, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y.).

Factor VII variants according to the invention may be produced using any appropriate host cell. In some embodiments, heterologous host cells are programmed to express the particular variant Factor VII from a recombinant gene. The host cells may be vertebrate, insect, fungal, or bacterial cells. The Factor VII variants may also be produced in transgenic animals or plants.

Examples of mammalian cell lines for use in the present invention are the COS-1 (ATCC CRL 1650), baby hamster kidney (BHK) and 293 (ATCC CRL 1573; Graham et al., J. Gen. Virol. 36:59-72, 1977) cell lines. A preferred BHK cell line is the tk³¹ ts13 BHK cell line (Waechter and Baserga, Proc. Natl. Acad. Sci. USA 79:1106-1110, 1982, incorporated herein by reference), hereinafter referred to as BHK 570 cells. The BHK 570 cell line has been deposited with the American Type Culture Collection, 12301 Parklawn Dr., Rockville, Md. 20852, under ATCC accession number CRL 10314. A tk⁻ ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61), DUKX cells (Urlaub and Chasin, Proc. Natl. Acad. Sci. USA 77:4216-4220, 1980) and CHO-DG44 cells (Urlaub et al. Cell 33: 405-412,1983.

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides there from are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are typically selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequences encoding Factor VII variants may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 438 The transformation of F. oxysporum may, for instance, be carried out as described by Malardier et al., 1989, Gene 78: 147-156. The transformation of Trichoderma spp. may be performed for instance as described in EP 244 234.

The present invention encompasses methods for producing glycosylation-disrupted Factor VII variants according to the invention. These methods are carried out by the steps of:
(a) culturing a cell expressing a glycosylation-disrupted Factor VII variant and
(b) recovering the Factor VII variant from the culture to obtain a preparation comprising the polypeptides.

The methods may further comprise purification and/or activation of the Factor VII variants.

Separation of Factor VII variant polypeptides from their cell of origin may be achieved by any method known in the art, including, without limitation, removal of cell culture medium containing the desired product from an adherent cell culture; centrifugation or filtration to remove non-adherent cells; and the like.

Optionally, Factor VII polypeptides may be further purified. Purification may be achieved using any method known in the art, including, without limitation, affinity chromatography, such as, e.g., on an anti-Factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J.-C. Janson and Lars Ryden, editors, VCH Publishers, New York, 1989. Following purification, the preparation preferably contains less than about 10% by weight, more preferably less than about ⁵% and most preferably less than about 1%, of non-Factor VII proteins derived from the host cell.

Factor VII and Factor VII variant polypeptides may be activated by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, Factor VII may be activated by passing it through an ion-exchange chromatography column, such as Mono Q® (Pharmacia) or the like. The resulting activated Factor VII may then be formulated and administered as described below.

Pharmaceutical Compositions and Methods of Use

The Factor VII variant polypeptides of the present invention may be used to treat any Factor VII-responsive syndrome, such as, e.g., bleeding disorders, including, without limitation, those caused by clotting factor deficiencies (e.g., haemophilia A and B or deficiency of coagulation factors XI or VII); by thrombocytopenia or von Willebrand's disease, or by clotting factor inhibitors, or excessive bleeding from any cause. The preparations may also be administered to patients in association with surgery or other trauma or to patients receiving anticoagulant therapy.

Pharmaceutical compositions comprising the Factor VII variants according to the present are primarily intended for parenteral administration for prophylactic and/or therapeutic treatment. Preferably, the pharmaceutical compositions are administered parenterally, i.e., intravenously, subcutaneously, or intramuscularly. They may be administered by continuous or pulsatile infusion.

Pharmaceutical compositions or formulations comprise a preparation according to the invention in combination with, preferably dissolved in, a pharmaceutically acceptable carrier, preferably an aqueous carrier or diluent. A variety of aqueous carriers may be used, such as water, buffered water, 0.4% saline, 0.3% glycine and the like. The Factor VII variants of the invention can also be formulated into liposome preparations for delivery or targeting to the sites of injury. Liposome preparations are generally described in, e.g., U.S. Pat. Nos. 4,837,028, 4,501,728, and 4,975,282. The compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc.

The concentration of Factor VII variant polypeptides in these formulations can vary widely, i.e., from less than about 0.5% by weight, usually at or at least about 1% by weight to as much as 15 or 20% by weight and will be selected primarily by fluid volumes, viscosities, etc., in accordance with the particular mode of administration selected.

Thus, a typical pharmaceutical composition for intravenous infusion may contain 250 ml of sterile Ringer's solution and 10 mg of the Factor VII variant polypeptide. Actual methods for preparing parenterally administrable compositions will be known or apparent to those skilled in the art and are described in more detail in, for example, Remington's Pharmaceutical Sciences, 18th ed., Mack Publishing Company, Easton, Pa. (1990).

The compositions containing the Factor VII variants of the present invention can be administered for prophylactic and/or therapeutic treatments. In therapeutic applications, compositions are administered to a subject already suffering from a disease, as described above, in an amount sufficient to cure, alleviate or partially arrest the disease and its complications. An amount adequate to accomplish this is defined as “therapeutically effective amount”. Effective amounts for each purpose will depend on the severity of the disease or injury as well as the weight and general state of the subject. In general, however, the effective amount will range from about 0.05 mg up to about 500 mg of the preparation per day for a 70 kg subject, with dosages of from about 1.0 mg to about 200 mg of the preparation per day being more commonly used. It will be understood that determining an appropriate dosage may be achieved using routine experimentation, by constructing a matrix of values and testing different points in the matrix.

Local delivery of the preparations of the present invention, such as, for example, topical application, may be carried out, e.g., by means of a spray, perfusion, double balloon catheters, stent, incorporated into vascular grafts or stents, hydrogels used to coat balloon catheters, or other well established methods. In any event, the pharmaceutical compositions should provide a quantity of the preparation sufficient to effectively treat the subject.

The present invention encompasses combined administration of an additional agent in concert with Factor VIIa or a Factor VIIa equivalent. In some embodiments, the additional agent comprises a coagulant, including, without limitation, a coagulation factor such as, e.g., Factor VIII, Factor IX (see, e.g., WO 02/062376), Factor V (see, e.g., PCT/DK02/00736), Factor XI, Factor XIII (see, e.g., WO 01/85198); prothrombin, or thrombin; or an inhibitor of the fibrinolytic system, such as, e.g., PAI-1 (see, e.g., PCT/DK02/00735), aprotinin, ε-aminocaproic acid (see, e.g., PCT/DK02/00752) or tranexamic acid (see, e.g., PCT/DK02/00751). Also included are inhibitors of tissue factor pathway inhibitor (TFPI inhibitors) (see, e.g., WO 01/85199); thrombin activatable fibrinolysis inhibitor (TAFI) (see, e.g., PCT/DK02/00734); protein C inhibitors (see, e.g., PCT/DK02/00737); thrombomodulin (see, e.g., PCT/DK02/00738); protein S inhibitors (see, e.g., PCT/DK02/00739); tissue plasminogen activator inhibitors (see, e.g., PCT/DK02/00740); α2-antiplasmin (see, e.g., PCT/DK02/00741); aprotinin (see, e.g., PCT/DK02/00742); and fibrinogen.

It will be understood that, in embodiments comprising administration of combinations of Factor VIIa with other agents, the dosage of Factor VIIa or Factor VIIa equivalent may on its own comprise an effective amount and additional agent(s) may further augment the therapeutic benefit to the patient. Alternatively, the combination of Factor VIIa or equivalent and the second agent may together comprise an effective amount for preventing late complications associated with trauma. It will also be understood that effective amounts may be defined in the context of particular treatment regimens, including, e.g., timing and number of administrations, modes of administrations, formulations, etc.

### BRIEF DESCRIPTION OF THE DRAWING

FIG. 1 is a Western blot comparing wild-type factor VII and glycosylation disrupted factor VII variants. Experimental details are as described in example 3.2. At the left are molecular mass markers and indications of the migration of Factor VII polypeptides containing 0, 1, or 2 N-linked core oligosaccharide chains.

FIG. 2 shows the full amino acid sequence of native (wild type) human coagulation Factor VII (SEQ ID NO:1).

The following examples are intended as non-limiting illustrations of the present invention.

### EXAMPLES

### Example 1

### Assays for Factor VII Biological Activity

The following experiments are performed to test the biological activity of Factor VII variants according to the invention.

In Vitro Hydrolysis Assay

Wild-type Factor VIIa and Factor VIIa variants (both hereafter referred to as “Factor VIIa”) may be assayed in parallel to compare directly their biological properties. The assay is carried out in a microtiter plate (MaxiSorp, Nunc, Denmark). The chromogenic substrate D-Ile-Pro-Arg-p-nitroanilide (S-2288, Chromogenix, Sweden), final concentration 1 mM, is added to Factor VIIa (final concentration 100 nM) in 50 mM Hepes, pH 7.4, containing 0.1 M NaCl, 5 mM CaCl₂ and 1 mg/ml bovine serum albumin. The absorbance at 405 nm is measured continuously in a SpectraMax™ 340 plate reader (Molecular Devices, USA). The absorbance developed during a 20-minute incubation, after subtraction of the absorbance in a blank well containing no enzyme is used to calculate the ratio between the activities of variant and wild-type Factor VIIa using the following formula:
Ratio=(A_{405 nm} Factor VIIa variant)/(A_{405 nm} Factor VIIa wild-type).

Based thereon, Factor VIIa variants with an activity comparable to or higher than native Factor VIIa may be identified, such as, for example, variants where the ratio between the activity of the variant and the activity of native Factor VII (wild-type FVII) is around, versus above 1.0.

The activity of Factor VIIa or Factor VIIa variants may also be measured using a physiological substrate such as Factor X, suitably at a concentration of 100-1000 nM, where the Factor Xa generated is measured after the addition of a suitable chromogenic substrate (eg. S-2765) (“the In Vitro Proteolysis Assay”). In addition, the activity assay may be run at physiological temperature.

In Vitro Proteolysis Assay

Wild-type Factor VIIa and Factor VIIa variant (both hereafter referred to as “Factor VIIa”) are assayed in parallel to directly compare their specific activities. The assay is carried out in a microtiter plate (MaxiSorp, Nunc, Denmark). Factor VIIa (10 nM) and Factor X (0.8 microM) in 100 microL 50 mM Hepes, pH 7.4, containing 0.1 M NaCl, 5 mM CaCl2 and 1 mg/ml bovine serum albumin, are incubated for 15 min. Factor X cleavage is then stopped by the addition of 50 microL 50 mM Hepes, pH 7.4, containing 0.1 M NaCl, 20 mM EDTA and 1 mg/ml bovine serum albumin. The amount of Factor Xa generated is measured by addition of the chromogenic substrate Z-D-Arg-Gly-Arg-p-nitroanilide (S-2765, Chromogenix, Sweden), final concentration 0.5 mM. The absorbance at 405 nm is measured continuously in a SpectraMax™ 340 plate reader (Molecular Devices, USA). The absorbance developed during 10 minutes, after subtraction of the absorbance in a blank well containing no FVIIa, is used to calculate the ratio between the proteolytic activities of variant and wild-type Factor VIIa according to the following formula:
Ratio=(A405 nm Factor VIIa variant)/(A405 nm Factor VIIa wild-type).
Thrombin Generation Assay:

The ability of Factor VII or Factor VII-related polypeptides or Factor VIII or Factor VIII-related polypeptides (e.g., variants) to generate thrombin can be measured in an assay comprising all relevant coagulation Factors and inhibitors at physiological concentrations and activated platelets (as described on p. 543 in Monroe et al. (1997) Brit. J. Haematol. 99, 542-547 which is hereby incorporated as reference).

Clot Assay:

The activity of the Factor VII polypeptides may also be measured using a one-stage clot assay (assay 4) essentially as described in WO 92/15686 or U.S. Pat. No. 5,997,864. Briefly, the sample to be tested is diluted in 50 mM Tris (pH 7.5), 0.1% BSA and 100 μL is incubated with 100 μL of Factor VII deficient plasma and 200 μL of thromboplastin C containing 10 mM Ca²⁺. Clotting times are measured and compared to a standard curve using a reference standard or a pool of citrated normal human plasma in serial dilution.

### Example 2

### Construction and Expression of Glycosylation-Disrupted Factor VII Variants

The following experiments were performed to produce glycosylation-disrupted Factor VII variants.

1. Construction of expression plasmids encoding human factor VII or glycosylation disrupted factor VII variants: Full-length human factor VII cDNA originating from the λHVII565 clone generated by Hagen et al. (Proc. Natl. Acad. Sci. USA, 83, 2412-2416, 1986) [accession no. M13232] was inserted into the BamH I/EcoR I sites of pcDN3.1+ (Invitrogen) to create the pTS8 plasmid. Constructs encoding disrupted factor VII variants were generated by site-directed mutagenesis of pTS8 using the QuickChange kit (Stratagene) as recommended by the manufacturer. The N145Q mutation was introduced with the 5′-TTCTAGAAAAAAGACAAGCCAGCAAACCCCAAGG-3′ (SEQ ID NO:2) forward primer (mutation in bold) and the complementary reverse primer, and the N322Q mutation was introduced with the 5′-GTGGGAGACTCCCCACAAATCACGGAGTACATG-3′ (SEQ ID NO:3) forward primer and the complementary reverse primer. Wild-type factor VII cDNA was subcloned from pTS8 into the Hind III/EcoR I sites of pMPSVHE (Artelt et al., Gene, 68, 213-219, 1988) to create the pTS39 plasmid. Likewise, mutated factor VII cDNA encoding the single or double N-glycosylation site knock-out mutations were inserted into the Mlu I/EcoR I sites of pMPSVHE to create the pCK711 plasmid encoding factor VII with the N145Q mutation (FVII-N145Q), the pCK712 plasmid encoding FVII-N322Q, and pCK713 encoding FVII-N145/322Q. The inserted factor VII genes were verified by DNA sequencing.

2. Generation of stable cells lines producing human factor VII or glycosylation disrupted factor VII variants: Chinese hamster ovary (CHO—K1) cells were co-transfected with pSV2-neo containing the neomycin resistance gene in combination with pTS39, pCK711, pCK712 or pCK713 using Lipofectamine (Invitrogen). Clones stably expressing the factor VII proteins were selected with 450 μg/ml G418. Resistant clones were screened by testing cell culture supernatants for factor VII by ELISA. This way, cell lines expressing wild-type human factor VII, FVII-N322Q, FVII-N145Q, or FVII-N145/322Q were established.

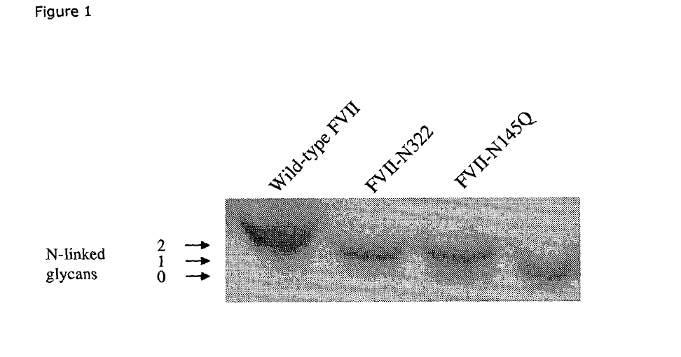

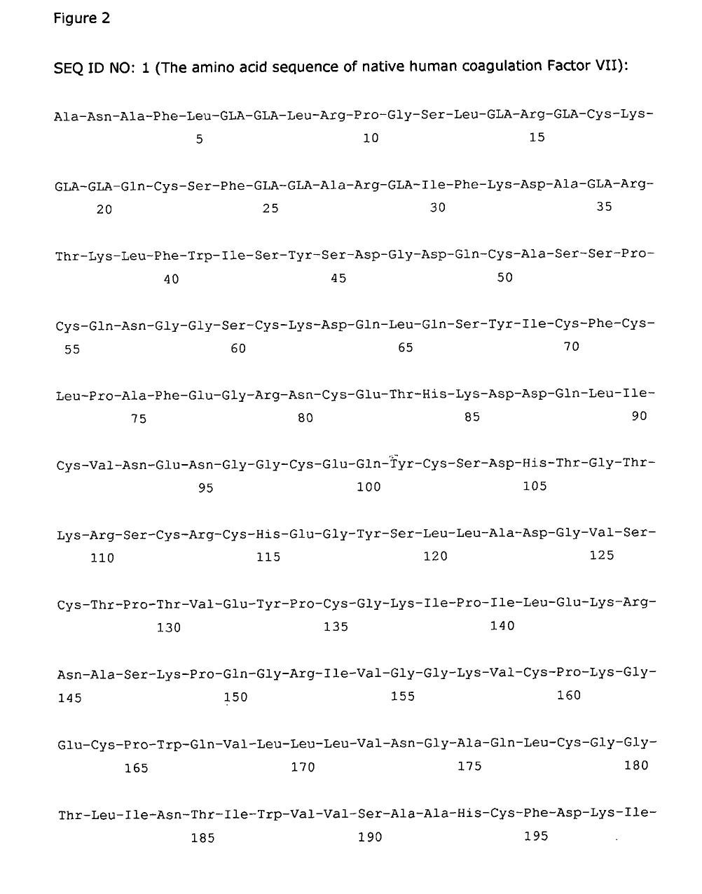

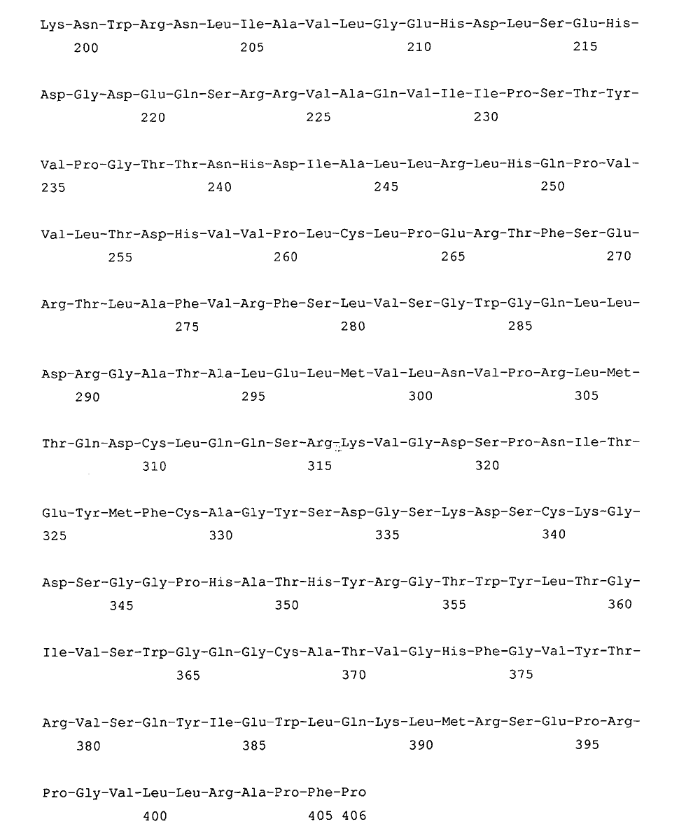

3. Comparison of wild-type factor VII and glycosylation disrupted factor VII variants by Western blotting: Medium from CHO—K1 derived cell lines expressing wild-type human factor VII, FVII-N322Q, FVII-N145Q, or FVII-N145/322Q was loaded on a SDS-PAGE gel and electrophoresed. The proteins in the gel were transferred to a PVDF membrane by electroblotting. Factor VII on the membrane was visualized by sequential incubation of the membrane with murine anti-FVII monoclonal antibody (clone FVII-4F9, Novo Nordisk) and HRP-conjugated rabbit anti-mouse IgG antibody (DAKO) followed by incubation with ECL Western Blotting Detection Reagent (Amersham Biosciences). Reading was carried out with a Las-1000 Luminescent image analyzer (Fujifilm). Wild-type factor VII and the three glycosylation-disrupted Factor VII variants were detected as distinct bands, demonstrating efficient secretion of all three glycosylation-disrupted Factor VII variants (FIG. 1). Each N-glycosylation knock-out mutation increased the electrophoretic mobility of the factor VII variant (FIG. 1). This confirms that the N-glycosylation sites of the factor VII variants were indeed disrupted as intended.

### Example 3

### Bioactivity of Glycosylation-Disrupted Factor VIIa

The following experiment was performed to test the bioactivity of glycosylation-disrupted Factor VIIa polypeptides.

Medium was collected from CHO—K1 derived stable clones transfected with expression plasmids containing the gene of wild-type human factor VII or the gene of human factor VII with one or two N-glycosylation knock-out mutations as described in Example 2 herein. The media were analyzed for factor VII content by enzyme-linked immunosorbent assay (ELISA) and for factor VII activity by clot assay. For the clot assay, media and factor VII standards diluted in 50 mM Pipes pH 7.2, 100 mM NaCl, 2 mM EDTA, and 1% BSA were mixed with an equal volume of factor VII deficient medium. The clotting time of each sample was determined in an ACL 300R (Instrumentation Laboratory) clotting instrument by addition of an equal volume of rabbit thromboplastin in 12.5 mM CaCl₂. The relationship between factor VII units and clotting time was determined with a standard curve, and the amount of units in each medium was calculated from the clotting time. The FVII activities of the recombinant wild-type and mutant factor VII were calculated by combining the ELISA and clot assay data. The results are shown in the below table:

| Factor VII protein | Activity (units/μg) |
|---|---|
| Wild-type | 1.6 |
| N145Q | 2.2 |
| N322Q | 2.5 |
| N145/322Q | 2.7 |

These results demonstrated that glycosylation-disrupted human factor VII exhibited increased activity compared to wild-type human factor VII.

### Example 4

### Comparison of the in vivo Kinetics of Glycosylation-Disrupted Factor VII with that of Wild-Type FVII

The following experiment was performed to compare the in vivo clearance of glycosylation-disrupted factor VIIa and wild-type factor VIIa

Male NMRI mice weighing approximately 30 g were injected in the tail vein with 1 mg/kg purified FVIIa-N145/322Q or wild-type FVIIa. Each of the 2 compounds was given to 13 or 9 mice as a single bolus injection. For each compound, blood samples were collected from the eyes of 2-3 anesthetized mice 0.08 h, 0.17 h, 0.33 h, 0.67 h, 1 h, 2h, 4 h, 6 h, and 8 h after injection. The blood was stabilized and examined for FVIIa by ELISA using specific standard curves for each of the 2 compounds. Parameters describing the clearance of the 2 compounds were calculated from the concentration-time profiles of the 2 compounds. The key parameters are shown in the below table and demonstrate that glycosylation-disrupted factor VIIa is cleared faster than wild-type factor VIIa.

| | Area under the curve | Half-life | Clearance |
|---|---|---|---|
| Compound | h × μg/ml | h | ml/h/kg |
| FVIIa-N145/322Q | 2.11 | 1.0 | 474 |
| Wild-type FVIIa | 8.32 | 1.6 | 120 |

references, including publications, patent applications, and patents, cited herein are hereby incorporated by reference to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The use of the terms “a” and “an” and “the” and similar referents in the context of describing the invention (especially in the context of the following claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

A description herein of an aspect or embodiment of the invention using terms such as “comprising”, “having,” “including,” or “containing” a particular element is intended to provide support for an aspect or embodiment of the invention that “consists of”, “consists essentially of”, or “substantially comprises” that particular element, unless otherwise stated or clearly contradicted by context.

This invention includes all modifications and equivalents of the subject matter recited in the claims appended hereto as permitted by applicable law.

1. A variant Factor VII polypeptide comprising at least one sequence alteration relative to the sequence of SEQ ID NO: 1, wherein said at least one alteration is selected from the group consisting of:
(i) substitution of N145 with any other amino acid except A;
(ii) substitution of N322 with any other amino acid except A or D; and
(iii) substitution of N145 with any other amino acid except A and independent substitution of N322 for any other amino acid except A or D;
(iv) substitution of N145 with any other amino acid and independent substitution of N322 for any amino acid except for A or D;
(v) substitution of N145 with any amino acid except A and independent substitution of N322 with any other amino acid; and
(vi) substitution at any position relative to the sequence of SEQ ID NO: 1, wherein said sequence alteration results in disruption of N-linked glycosylation at N145, N322, or both N145 and N322 and wherein said sequence alteration is not at positions 145 or 322.

2. A polypeptide according to claim 1, wherein said alteration which results in disruption of N-linked glycosylation comprises N145Q; N322Q; or, N145Q and N322Q.

3. A polypeptide according to claim 1, wherein said alteration which results in disruption of N-linked glycosylation is at position 146, 323, or both 146 and 323 relative to the sequence of SEQ ID NO:1.

4. A polypeptide according to claim 1, wherein said alteration which results in disruption of N-linked glycosylation is at position 147, 324, or both 327 and 324 relative to the sequence of SEQ ID NO:1.

5. A polypeptide according to claim 1, wherein said alteration which results in disruption of N-linked glycosylation is at position 148, 325, or both 148 and 325 relative to the sequence of SEQ ID NO:1.

6. A polypeptide according to claim 1, wherein said alterations which results in disruption of N-linked glycosylation are selected from K143N/N145T and K143N/N145T/R315N/V317T.

7. A polypeptide according to claim 1, further comprising a sequence alteration selected from the group consisting of: R152E; S344A; L305V; L305V/M306D/D309S; L305T, L305T, F374P, V158T/M298Q, V158D/E296V/M298Q, K337A, M298Q, V158D/M298Q, L305V/K337A, V158D/E296V/M298Q/L305V, V158D/E296V/M298Q/K337A, V158D/E296V/M298Q/L305V/K337A, K157A, E296V, E296V/M298Q, V158D/E296V, V158D/M298K, and S336G, L305V/K337A, L305V/V158D, L305V/E296V, L305V/M298Q, L305V/V158T, L305V/K337A/V158T, L305V/K337A/M298Q, L305V/K337A/E296V, L305V/K337A/V158D, L305V/V158D/M298Q, L305V/V158D/E296V, L305V/V158T/M298Q, L305V/V158T/E296V, L305V/E296V/M298Q, L305V/V158D/E296V/M298Q, L305V/V158T/E296V/M298Q, L305V/V158T/K337A/M298Q, L305V/V158T/E296V/K337A, L305V/V158D/K337A/M298Q, L305V/V158D/E296V/K337A-FVII, L305V/V158D/E296V/M298Q/K337A, L305V/V158T/E296V/M298Q/K337A, S314E/K316H, S314E/K316Q, S314E/L305V, S314E/K337A, S314E/V158D, S314E/E296V, S314E/M298Q, S314E/V158T, K316H/L305V, K316H/K337A, K316H/V158D, K316H/E296V, K316H/M298Q, K316H/V158T, K316Q/L305V, K316Q/K337A, K316Q/V158D, K316Q/E296V, K316Q/M298Q, K316Q/V158T, S314E/L305V/K337A, S314E/L305V/V158D, S314E/L305V/E296V, S314E/L305V/M298Q, S314E/L305V/V158T, S314E/L305V/K337A/V158T, S314E/L305V/K337A/M298Q, S314E/L305V/K337A/E296V, S314E/L305V/K337A/V158D, S314E/L305V/V158D/M298Q, S314E/L305V/V158D/E296V, S314E/L305V/V158T/M298Q, S314E/L305V/V158T/E296V, S314E/L305V/E296V/M298Q, S314E/L305V/V158D/E296V/M298Q, S314E/L305V/V158T/E296V/M298Q, S314E/L305V/V158T/K337A/M298Q, S314E/L305V/V158T/E296V/K337A, S314E/L305V/V158D/K337A/M298Q, S314E/L305V/V158D/E296V/K337A, S314E/L305V/V158D/E296V/M298Q/K337A, S314E/L305V/V158T/E296V/M298Q/K337A, K316H/L305V/K337A, K316H/L305V/V158D, K316H/L305V/E296V, K316H/L305V/M298Q, K316H/L305V/V158T, K316H/L305V/K337A/V158T, K316H/L305V/K337A/M298Q, K316H/L305V/K337A/E296V, K316H/L305V/K337A/V158D, K316H/L305V/V158D/M298Q, K316H/L305V/V158D/E296V, K316H/L305V/V158T/M298Q, K316H/L305V/V158T/E296V, K316H/L305V/E296V/M298Q, K316H/L305V/V158D/E296V/M298Q, K316H/L305V/V158T/E296V/M298Q, K316H/L305V/V158T/K337A/M298Q, K316H/L305V/V158T/E296V/K337A, K316H/L305V/V158D/K337A/M298Q, K316H/L305V/V158D/E296V/K337A, K316H/L305V/V158D/E296V/M298Q/K337A, K316H/L305V/V158T/E296V/M298Q/K337A, K316Q/L305V/K337A, K316Q/L305V/V158D, K316Q/L305V/E296V, K316Q/L305V/M298Q, K316Q/L305V/V158T, K316Q/L305V/K337A/V158T, K316Q/L305V/K337A/M298Q, K316Q/L305V/K337A/E296V, K316Q/L305V/K337A/V158D, K316Q/L305V/V158D/M298Q, K316Q/L305V/V158D/E296V, K316Q/L305V/V158T/M298Q, K316Q/L305V/V158T/E296V, K316Q/L305V/E296V/M298Q, K316Q/L305V/V158D/E296V/M298Q, K316Q/L305V/V158T/E296V/M298Q, K316Q/L305V/V158T/K337A/M298Q, K316Q/L305V/V158T/E296V/K337A, K316Q/L305V/V158D/K337A/M298Q, K316Q/L305V/V158D/E296V/K337A, K316Q/L305V/V158D/E296V/M298Q/K337A, K316Q/L305V/V158T/E296V/M298Q/K337A, F374Y/K337A, F374Y/V158D, F374Y/E296V, F374Y/M298Q, F374Y/V158T, F374Y/S314E, F374Y/L305V, F374Y/L305V/K337A, F374Y/L305V/V158D, F374Y/L305V/E296V, F374Y/L305V/M298Q, F374Y/L305V/V158T, F374Y/L305V/S314E, F374Y/K337A/S314E, F374Y/K337A/V158T, F374Y/K337A/M298Q, F374Y/K337A/E296V, F374Y/K337A/V158D, F374Y/V158D/S314E, F374Y/V158D/M298Q, F374Y/V158D/E296V, F374Y/V158T/S314E, F374Y/V158T/M298Q, F374Y/V158T/E296V, F374Y/E296V/S314E, F374Y/S314E/M298Q, F374Y/E296V/M298Q, F374Y/L305V/K337A/V158D, F374Y/L305V/K337A/E296V, F374Y/L305V/K337A/M298Q, F374Y/L305V/K337A/V158T, F374Y/L305V/K337A/S314E, F374Y/L305V/V158D/E296V, F374Y/L305V/V158D/M298Q, F374Y/L305V/V158D/S314E, F374Y/L305V/E296V/M298Q, F374Y/L305V/E296V/V158T, F374Y/L305V/E296V/S314E, F374Y/L305V/M298Q/V158T, F374Y/L305V/M298Q/S314E, F374Y/L305V/V158T/S314E, F374Y/K337A/S314E/V158T, F374Y/K337A/S314E/M298Q, F374Y/K337A/S314E/E296V, F374Y/K337A/S314E/V158D, F374Y/K337A/V158T/M298Q, F374Y/K337A/V158T/E296V, F374Y/K337A/M298Q/E296V, F374Y/K337A/M298Q/V158D, F374Y/K337A/E296V/V158D, F374Y/V158D/S314E/M298Q, F374Y/V158D/S314E/E296V, F374Y/V158D/M298Q/E296V, F374Y/V158T/S314E/E296V, F374Y/V158T/S314E/M298Q, F374Y/V158T/M298Q/E296V, F374Y/E296V/S314E/M298Q, F374Y/L305V/M298Q/K337A/S314E, F374Y/L305V/E296V/K337A/S314E, F374Y/E296V/M298Q/K337A/S314E, F374Y/L305V/E296V/M298Q/K337A, F374Y/L305V/E296V/M298Q/S314E, F374Y/V158D/E296V/M298Q/K337A, F374Y/V158D/E296V/M298Q/S314E, F374Y/L305V/V158D/K337A/S314E, F374Y/V158D/M298Q/K337A/S314E, F374Y/V158D/E296V/K337A/S314E, F374Y/L305V/V158D/E296V/M298Q, F374Y/L305V/V158D/M298Q/K337A, F374Y/L305V/V158D/E296V/K337A, F374Y/L305V/V158D/M298Q/S314E, F374Y/L305V/V158D/E296V/S314E, F374Y/V158T/E296V/M298Q/K337A, F374Y/V158T/E296V/M298Q/S314E, F374Y/L305V/V158T/K337A/S314E, F374Y/V158T/M298Q/K337A/S314E, F374Y/V158T/E296V/K337A/S314E, F374Y/L305V/V158T/E296V/M298Q, F374Y/L305V/V158T/M298Q/K337A, F374Y/L305V/V158T/E296V/K337A, F374Y/L305V/V158T/M298Q/S314E, F374Y/L305V/V158T/E296V/S314E, F374Y/E296V/M298Q/K337A/V158T/S314E, F374Y/V158D/E296V/M298Q/K337A/S314E, F374Y/L305V/V158D/E296V/M298Q/S314E, F374Y/L305V/E296V/M298Q/V158T/S314E, F374Y/L305V/E296V/M298Q/K337A/V158T, F374Y/L305V/E296V/K337A/V158T/S314E, F374Y/L305V/M298Q/K337A/V158T/S314E, F374Y/L305V/V158D/E296V/M298Q/K337A, F374Y/L305V/V158D/E296V/K337A/S314E, F374Y/L305V/V158D/M298Q/K337A/S314E, F374Y/L305V/E296V/M298Q/K337A/V158T/S314E, F374Y/L305V/V158D/E296V/M298Q/K337A/S314E; R152E, S344A; P11Q/K33E, T106N, V253N, R290N/A292T, G291N, R315N/V317T, and K143N/R315N/V317T.

8. A polypeptide according to claim 1, wherein said polypeptide has a decreased half-life as compared to wild type human FVIIa.

9. A pharmaceutical formulation comprising a polypeptide according to claim 1 and a pharmaceutically acceptable carrier or excipient.

10. A method for treating a Factor VII-responsive syndrome, which comprises administering to a patient in need of such treatment a therapeutically effective amount of a formulation according to claim 9.

11. A kit comprising a therapeutically effective amount of a polypeptide according to claim 1.


SEQUENCE LISTING (3 sequences)

SEQ ID NO: 1 - protein, 406 aa, Homo sapiens
  MISC_FEATURE (1)..(406): Xaa means 4-carboxyglutamic acid (gamma-carboxyglutamate)
ANAFLXXLRPGSLXRXCKXXQCSFXXARXIFKDAXRTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 2 - DNA, 34 bp, Unknown
  DNA primer for preparation of N145Q-FVII
ttctagaaaaaagacaagccagcaaaccccaagg

SEQ ID NO: 3 - DNA, 33 bp, Unknown
  DNA primer for preparation of N322Q-FVII
gtgggagactccccacaaatcacggagtacatg